(19) United States
(12) Patent Application Publication
(10) Pub. No.: US 2017/0367522 A1
(43) Pub. Date: Dec. 28, 2017

(54) BEER POST-MATURATION CUTOMISATION APPLIANCE

(71) Applicant: Marcos Valadao Abi Ackel, Belo Horizonte (BR)

(72) Inventors: Marcos Valadao Abi Ackel, Belo Horizonte (BR); Wilfred Brandt, Belo Horizonte (BR); Rubens Valadao Abi Ackel, Belo Horizonte (BR); Emanuel Manfred Freire Brandt, Belo Horizonte (BR)

(21) Appl. No.: 15/537,298

(22) PCT Filed: Dec. 2, 2015
(86) PCT No.: PCT/BR2015/050230

§ 371 Date: Jun. 16, 2017

(30) Foreign Application Priority Data
Dec. 19, 2014 (BR) BR1020140329080

(51) Int. Cl. A47J 31/41; A47J 31/40; C12C 12/00; B67D 1/02; G05D 11/03; C12G 3/06; C12P 1/00

(52) U.S. Cl. CPC A47J 31/41; C12C 12/006; A47J 31/407; B67D 1/025; A47J 31/402; C12G 3/06; C12P 1/00; G05D 11/03

(57) ABSTRACT

This invention patent applies to the manufacture and beer consume areas and is about a device for the customization of postmaturation beer allowing to add ingredients on already done beers, in order to change its visual and sensory characteristics, according to the adjustments executed by the user or by a recipe that can be programmed on the device. Referring to the domestic customization device for the visual and sensory characteristics of the already done beers, having the objective of altering it, according to the user parameters, and the characteristics of the already done beers, inside several conditions, with the introduction of supplies such as liquids, syrups and having the possibility of cold infusion.

### CROSS-REFERENCE TO RELATED APPLICATIONS(S)

The present application claims priority to International Application No. PCT/BR2015/050230 filed on Dec. 2, 2015, entitled “APARELHO PARA CUSTOMIZA [symbol] ÃO DE CERVEJAS P [symbol] S-MATURA [symbol] ÃO (BEER POST-MATURATION CUSTOMISATION APPLIANCE)” the entire disclosure of which is incorporated by reference herein.

### BACKGROUND OF THE INVENTION

### 1. Description of Related Art

Currently, there are kits and equipment of homemade production beers, which allow the users to manufacture their own beer, composed mainly of malt, hop and yeasts and associates, controlling the production parameters of the drink. This process, however, by its own nature, takes some days, contrary to the machines found on the market, not applied to beer manufacture, because it is composed by already done beers in variable mixtures and quantities, also several ingredients which will function to change the characteristics of the beverage, such as appearance (color, foam and transparency), odor, taste and tactile sensations caused on the mouth (body, texture, carbonation, alcoholic strength, etc.).

MU8802350-8—“Constructive disposition introduced on a mixer machine and juice and associate dispenser”—referring to a juice mixer and dispenser machine, used on commercial establishment, which allows the

“Constructive disposition introduced on a mix machine and juice dispenser and correlated”—related to a mix machine and juice dispensers, used on commercial establishments allowing to obtain juices and associated from the instantaneous and homogenous mix of the pulp concentrated with cooled water; formed by a cabined with an activation panel with several buttons and multiple dosage nozzle; overlaid to the panel previewed as a main opening with an articulable tamp, on the extreme inferior part of the cabinet a trimmer is previewed; the internal portion and inferior of the cabinet which is previewed as a controller space, this storage space presents a water cooler reservoir with articulable tamp and a water pump promoting the water release through a distribution hose; the liquid storage space presents a cooled receptacle receiving multiple pump concentrated gallons which are adapted to individual distribution mechanisms, being that every individual mechanism has a peristaltic pump and a conduction hose. The equipment has the objective of producing juices from the fruit pulp water dilution, specific for commercial establishment, not needing to add ingredients and/or visual characteristics and/or sensory in a controlled form if an already done beer.

PI8206313—“System of after mix beverage, construction of a cabinet, improvement of an after mix distribution system of the beverages and a process to load recipients of supply of the syrup and recipients of CO² supply” corresponding to U.S. Pat. No. 4,493,441, on Nov. 12, 1981 “Portable post-mix beverage dispenser unit”—referring to an equipment that, even when having the portable characteristic and not having access to commercial establishments, being a device that has as an objective to dilute syrups on water or carbonated water for the production of after mix beverage. This PI is directed towards the production of sodas and juices from the concentrated dilution (syrup) on water, not needing to add a controlled form of ingredients and/or visual and/or sensory characteristics to an already done beer.

PI1102551-4 “Device of drink mix, and, method of beverage mix” corresponding to the patent DE 102010029125.0, on May 19, 2010—“Vorrichtung and Verfahren zum Ausmischen von Getränken” referring to a device and a mix method of a beverage due to the fact that the aquatic mix phase, in which the components of the recipe that are necessary for the beverage mix are contained and have a water level higher to the mixed beverage, is collected on a tank, the mixed phase can add specifically a beverage mix with excessive concentration, in order to adjust the final concentration. The mix phase contains preferentially proportions returned to the prepared mixed beverage. Belonging to an industrial equipment of mix beverage preparation that will be, later on, packaged and commercialized, not adding any sort of controlled form of ingredients and/or visual and/or sensory characteristics to an already done beer.

PI0608648-9—“Independent beverage distribution machine” corresponding to the patent PCT/EP2006/002268, on Mar. 13, 2006, “Standalone drink dispensing machine” referring to the machine for the preparation and distribution of extraction beverages, under pressure, of a feeding substance contained on the capsule that might function independently in order to distribute several beverage successively. Comprehending and extraction module for the receiving of a capsule, the water reservoir thermic isolated for the supply of the extraction module, a pump and the power supply devices comprehending an electric accumulator of low voltage configured to supply the pump; the reservoir comprehends the heating devices, including and energized electric element by the accumulator. Specifically regarding the equipment that has as an objective to extract flavors and odors of pulverized solids with the use of hot water. This invention patent does not archive the extraction or infusion of hot water.

A PI0708600-8—“Product dispenser system, and beverage dispenser system” according to U.S. Pat. No. 7,757,896, on Mar. 6, 006, “Beverage dispensing system”—describes a product dispenser including at least one source of macro ingredient, at least one source of positioned micro ingredient over the dispenser, a diluent source, a dispenser valve, several pumps or dosage devices, and a user interface. The user interface receives a request for the type of product and instructs the pumps or dosage devices to dispense a type and a pre-determined relation of macro ingredient, of micro ingredient, and the diluent to the dispenser valve to a pre-determined flow. Is part of the equipment being used on industrial and commercial establishment which has as an objective diluting syrups on water or carbonated water for the production of beverages after the mix. The difference related to this patent request is that this equipment does not have the intention of customizing an already existing beverage, but to produce a beverage form the dilution of syrups and juice concentrated, on a diluent (generally on water), on portions of approximately 3:1 to 6:1, being able to still include micro ingredients which complement the beverage composition, with dilution of approximately 1:10 or more. In all the cases the equipment described on the patent PI0708600-8 has the objective of reconstituting of the ingredients on a diluent (generally water) for the production of a beverage, but not the customization of the already existing beverage. Differently of this equipment, this invention patent does not use diluent and the beverage to be consumed already exists before the start of the customization.

PI0708609-1—“Dispenser system and product preparation method” corresponding to US2007063036, on Mar. 10, 2007, “Juice Dispensing System”—describing the product mix device including the ingredient combination chamber and the agitation form positioned over the ingredient combination chamber including the diluent entry, a macro-ingredient entry quantity, an entry and exist of the micro-ingredient quantity. Regarding the equipment to produce juices through the dilution of fruit pulp water, specific for the industrial and commercial establishment. A processor of electronic dilution. Therefore, PI0708600-8 previously mentioned, the equipment described at PI0708609-1 has the objective of producing a beverage from the concentrated (such as fruit pulps and syrups) to be diluted or rebuilt, but is not an equipment which has the objective of customizing the already existing beverage for personal use and individualized as a present patent request.

PI9006775-4—“System and process to apply a concentrated mix and a diluent, system to apply a liquid and a set of flow control” corresponding to U.S. Pat. No. 9,002,577, on May 11, 1990, “Multiflavor beverage dispenser control valve assembly and ratio control system therefor” referring to the equipment having as an objective to execute the electronic control of beverage mix, to be used alongside the beverage preparation machines, specifically regarding the measurement systems and unique algorithms, regarding the control systems. This system/process is also destined to mix the concentrated to a diluent for a beverage formulation after the dilution of the constituents.

PI9306480-2—“Device and process to provide volume of concentrated dosage and diluent on controlled proportion of a mixes station on a beverage dispenser to produce a post mixed beverage and a dispenser valve of the post mix beverage” corresponding to U.S. Pat. No. 9,305,189, on Jun. 2, 1993, “Device for volumetric dosing of products”—referring to a dispenser valve of the post mix beverage to dispense syrup and soda on the desired relation without the need of flow controls to adjust periodically. The valve includes a flowmeter of the soda flow and the dosage pump of the syrup including a chamber inside the ceramic sleeve with two distal ends and an a double action ceramic piston assembled for the alternative sliding course inside the sleeve; a valve control system where the solenoid controls the syrup flow through a pump, answering the issuing signals through the flowmeter; the valve includes a syrup flow regulator and might include a sole tax control of the flow to any desired value. Referring specifically to the flow tax control to be used as part of the syrup diluting devices on water or carbonated water for the post mix beverage production.

PI0409070-5—“Method and apparatus for producing a tea beverage employing a continuous mixing chamber” corresponding to US2004008836 on Mar. 23, 2004 “Method and apparatus for producing a tea beverage employing a continuous mixing chamber”—referring to a mixed chamber in order to produce a tea base non-carbonated beverage, where the tea base beverage is susceptible to the growth of dangerous microorganisms, comprehending a hot water concentrated feeder line; a cold water feeder line with verification valve. Also referring to the production method of a non-carbonated tea base beverage, comprehending the phases of: hot water continuous feed, the tea concentrated and the cold water inside a mix chamber; mix continuously the tea concentrated, the hot and cold water on the mix chamber; and distribute it continuously on the tea base beverage in order to execute the tea having the objective of extracting flavors and odors of pulverized solids, with the use of hot or cold water, not adding ingredients and/or visual characteristics and/or sensory on a controlled form of an already done beer.

The patents mentioned above refers to two main groups: equipment destined to execute mixes between a concentrate and a diluent, generally water or carbonated water, and these include the ones with syrup and the ones with fruit pulps as a concentrate; the final product of these equipment is specifically a beverage after mix, which only actually exists after the mix. These equipment are normally destined for the use on industrial and commercial installations; equipment destined to extract flavors and odors of pulverized solids with the use of hot or cold water, and these include coffeemakers and tea producer equipment.

The main difference of this patent with these two groups is on its structure, related to its finality and function, because this patent does not produce the beverage from the dilution of syrups or pulps, which will be drunk after the mix. This patent adds ingredients and modifies the visual and sensory characteristics of already done beers for consumption. Other substantial difference is the fact that this patent does not use water as a diluent to be mixed to the concentrate parts and to form the beverage, being the device for customization the flavor of already done beers (postmatured), which will be tempered and mixed.

### SUMMARY OF THE INVENTION

This invention patent is applied to the production and consume area of the beer and refers to device for the postmaturing device for beer customization which allows to increase the ingredients on beers which are ready to be consumed, in order to modify its visual and sensory characteristics, according to the adjustments executed by the user or by a recipe that can be programmed on the device.

This invention is a domestic device of customization of visual and sensory characteristics of ready beers, having as an objective to modify, according to the user parameters, the beer characteristics of beers which are ready, inside several conditions, with the introduction of supplies such as liquids, syrups and with the possibility of executing the infusion on a cold situation.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a schematic of the postmaturing device for beer customisation, according to an embodiment of the present invention.

### DETAILED DESCRIPTION OF PREFERRED EMBODIMENTS

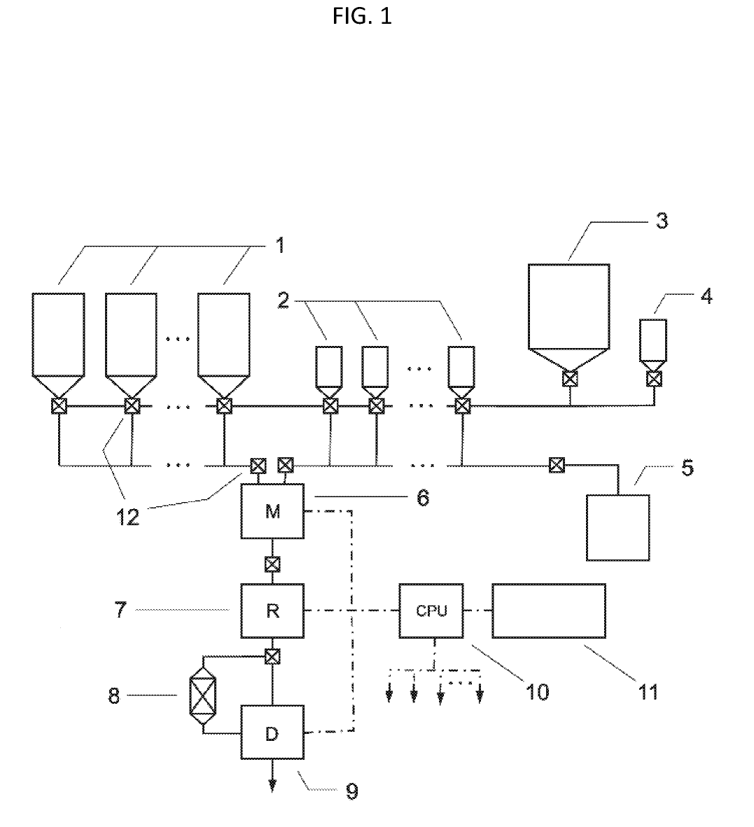

Preferred embodiments of the present invention and their advantages may be understood by referring to FIG. 1, wherein like reference numerals refer to like elements.

This device presents the following advantages: allows to add ingredients and visual and sensory characteristics to already done beers; allows the users to appreciate their favorite customized beer in a few minutes; allows the customization to be executed through pre-established dosages or even chosen dosages and executed freely by the user; allows to receive information about dosages (recipes) executed by third parties and that can be programmed on the device by the user; allows more practicality, safety and commodity on the consume of the customized beers.

This device might be able to present multiple external configuration of the cabinet, might have its components comprehended through the FIG. 1 which shows the disposition of the device components.

According to FIG. 1, the device presents the following components: reservoirs (1) replaceable for already done beers which serve as a base for the preparation of the customized beer, jars (2) replaceable ingredients containing individually one type of ingredient to be added to the selected mix of the base-beer that are on the reservoirs (1); the water reservoir (3) for the cleaning operation and sanitization of the system or extraction of several ingredients (2); reservoir of the sanitization/disinfectant (4) used for the depollution of the system and that can be diluted or not with water; waste reservoir (5) to accumulate the liquids of waste cleaning; mixer (6) that homogenizes the customized beer formula to be prepared; cooler (7) to cool the beer already customized, taking the beverage to the consume desired temperature; infuser cold hop reservoir (8) that allows the customized beer contact with the hop in order to add specific flavors or odors to the mix; dispenser (9) that storages and serves the customized beer to the user; CPU (10), Processing Central Unit, controlling all the equipment processes, receiving instructions and showing the internal status at the control panel (11); control panel (11) necessary for the whole information and equipment programing, also showing the internal or intermediary status of the customization phases; electrical valves (12) controlling the liquid flow (beers, ingredients, water, etc.) or gas injections inside the equipment, activated electrically, preferentially controlled by the CPU (10).

In an embodiment, the device has multiple receptacles to host the beer reservoirs (1). The jars (2) might contain, for example, malt or hop extracts, hops in the solid form, as well as specific flavors or odors or even other type of ingredients, such as CO² (Carbon Dioxide) for the gasification, or N² (Nitrogen) in order to improve the foam consistency.

This invention allows the user to charge the equipment with one or many type of beers ready according to the receptacles of the reservoirs (1), several ingredient bottles (2) and the cold hop infusion reservoir (8), these components might present identifiers such as bar codes or “chips”, in order to identify the equipment in which the receptacle is connected to each beer or ingredient. Through a control panel (11) the user might compose a mix, specifying the proportions or measures in order to compose the desired customized beer composition, including volume and temperature.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A method for customizing postmaturation beers comprising the steps of:
a. allowing the addition and special combination of ingredients to post-mature base-beers as a consequence of the structural and functional configuration of the system components, formed by its disposition and/or sequence of replaceable reservoirs (2) which release its contents on a mixer (6);
b. auctioning through a central processing unit (10), resulting on customized beer that will be optionally forwarded to the infuser reservoir cold hop (8), and consequently to the dispenser (9), being able to also contain a cooler for customized beer (7).

2. The method of customizing postmaturation beers of claim 1, further comprising the steps of presenting the following components: reservoirs (1) can be changed by already done beers which are base for the manufacture of the customized beers, reservoirs or jars (2) of the replaceable ingredients containing individually a sort of ingredient to be added to the selected mix of base-beers on the reservoirs (1); water reservoir (3) for the cleaning operations and system sanitization or extraction of several ingredients (2); sanitizing and/or disinfectant reservoir (4) used for the depollution of the system and that can be diluted or not on water; waste reservoir (5) to save the residual cleaning liquids; mixer (6) which homogenize the manufacture of the customized beer formula; cooler (7) to cool the already customized beer, taking the beer to the desired consume temperature; infusion reservoir cold hop (8) allowing contact with the customized beer and the hop in order to add specific flavors and odors; dispenser (9) which stores and serves the customized beer to the user; CPU (10), Central Processing Unit, controlling all the equipment process, receiving commands and showing the internal status on the control panel (11); control panel (11) if necessary for the whole operation and equipment programing, also showing the internal or intermediary states of the customization phases; electrical valves (12) controlling the liquid flow (beers, ingredients, water, etc.) or injection of gases inside the equipment, added electronically, preferentially executed by the CPU (10).

3. The method of customizing postmaturation beers of claim 2, further comprising the step of presenting multiple beer receptacles (1) and multiple reservoirs or jars (2) that might contain malt or hop extracts, hops on the solid form, as well as specific flavors and odors or even other sort of ingredients, such as CO2 (Carbone Dioxide) for the gasification, or N2 (Nitrogen) to create the texture of foam.

4. The method of customizing postmaturation beers of claim 3, further comprising the step of using the equipment with one or more already done beers on the receptacles of the reservoirs (1), and several ingredients jars (2) and the infuser reservoir cold hop (8) these components might present identifiers such as bar codes or chips in a way that the equipment is able to identify in which receptacle is connected to each beer or ingredient; through the control panel (11) the user will be able to comprise the desired beer customization, as well as the volume and temperature.
